(19) United States
(12) Patent Application Publication
(10) Pub. No.: US 2026/0294447 A1
(43) Pub. Date: Oct. 1, 2026

(54) Expandable Acetabular Reamer

(71) Applicant: David A. Crawford, Upper Arlington, OH (US)

(72) Inventor: David A. Crawford, Upper Arlington, OH (US)

(21) Appl. No.: 19/384,151

(22) Filed: Nov. 10, 2025

Related U.S. Application Data
(60) Provisional application No. 63/777,403, filed on Mar. 25, 2025.
(60) Provisional application No. 63/905,813, filed on Oct. 27, 2025.

(51) Int. Cl. A61B 17/16; A61B 17/00

(52) U.S. Cl. CPC A61B 17/1666; A61B 2017/00473

(57) ABSTRACT

A reamer for forming cavities in bone includes a housing, a support member extending coaxially within and supporting the housing, a scroll gear extending from the support member, and a first group of cutting blades. Each cutting blade in the first group of cutting blades includes a plurality of teeth extending toward and engaging the scroll gear such that rotation of the scroll gear causes each cutting blade in the first group of cutting blades to move. Even more, each cutting blade in the first group of cutting blades at least partially extends through the housing in a cutting position. The reamer may further include a second group of cutting blades wherein each of the cutting blades in the second group of cutting blades extends through the housing. The first and second groups of cutting blades define a substantially hemispherical cutting area having an outer diameter.

### RELATED APPLICATIONS

This application claims priority from U.S. Provisional Patent Application Ser. Nos. 63/777,403 filed on Mar. 25, 2025, and 63/905,813 filed on Oct. 27, 2025, the disclosures of which are incorporated herein by reference in their entirety.

### FIELD OF THE INVENTION

This document relates generally to power-driven devices for use in hip arthroplasty surgery, and more specifically to expandable acetabular reamers.

### BACKGROUND OF THE INVENTION

In total hip arthroplasty surgery, a power-driven reamer is utilized to prepare a cavity in the acetabulum for receiving an implant. The acetabular reamer typically has a substantially hemispherical cutting head for forming the cavity. A surgeon may utilize one or more fixed-size acetabular reamers within each procedure. Up to ten or more such individual reamers may be available with each reamer having a fixed cutting diameter (e.g., 52 mm, . . . 57 mm, etc.). The particular reamer size is chosen depending on a desired cavity size and, as suggested above, multiple reamers may be utilized during a procedure to gradually or incrementally create and increase the size of a cavity. The size of the final cavity depends in part on the surgeon's preference and the manner in which the implant will be anchored.

In one popular approach, the formed cavity or socket is under-sized to receive a slightly larger acetabular component or shell of the implant. In this manner, the acetabular shell may be press fit into the prepared socket providing a stable implant with reduced bone removal. The acetabular shell is typically made of metal (e.g., titanium, cobalt-chromium, or tantalum) for strength purposes and may include a porous and/or coated outer surface to encourage bone engagement and ingrowth to ensure long-term fixation. Of course, other procedures may utilize as few as one fixed-size reamer and different methods of anchoring the implant including screw(s) and/or cement fixation. An acetabular liner is commonly positioned within the seated acetabular shell providing a smooth surface for engaging a femoral head component of the implant.

In addition to being generally hemispherical in shape, acetabulum reamer cutting heads typically incorporate a “cheese grater” type cutting mechanism. Such mechanisms typically have multiple cutting members that engage and ream the bone during use. Open-fluted reamers, i.e., reamers using an open-fluted type cutting mechanism, are commonly used for more aggressive bone removal while closed-edge reamers are used for a smoother, more controlled shaping process. While such “cheese grater” cutting mechanisms are effective in forming a cavity, the fixed-size reamers have certain shortcomings, including: (1) dulling of the edges of the cutting members; (2) extended procedure durations due to the time incurred in exchanging multiple fixed diameter reamers; and (3) suboptimal cleaning and sterilization cycles given the geometries of the reamer cutting heads.

These shortcomings can cause suboptimal procedure results including, for example, reduced bone engagement which can result in long-term fixation issues. Such results can be caused by dulling that occurs due to the small, thin nature of the “cheese grater” cutting head edges. Under high torque and repeated force against dense bone, the small cutting edges can deform, chip, and/or become rounded off. Continued use of dull or dulling blades can cause increased heat generation causing the reamer to burnish rather than cut the bone which can reduce the mechanical interlock potential between the socket and the acetabular shell.

Accordingly, an acetabular reamer is needed that utilizes a cutting mechanism having sufficient mass and edge durability to minimize detrimental effects caused by dulling while providing on-the-fly adjustability without the need to remove and replace the reamer cutting head.

### SUMMARY OF THE INVENTION

In accordance with the purposes and benefits described herein, a reamer for forming cavities in bone is provided. The reamer may be broadly described as including a housing, a support member extending coaxially within and supporting the housing, a scroll gear extending from the support member, and a first group of cutting blades. Each cutting blade in the first group of cutting blades may include a plurality of teeth extending toward and engaging the scroll gear such that rotation of the scroll gear causes each cutting blade in the first group of cutting blades to move dependent upon a direction of rotation of the scroll gear, and each cutting blade in the first group of cutting blades at least partially extends through the housing in a cutting position.

In another possible embodiment, the reamer may further include a second group of cutting blades, wherein each of the cutting blades in the second group of cutting blades extends through the housing.

In yet another possible embodiment, each of the cutting blades in the second group of cutting blades is fixed in position relative the housing.

In still another possible embodiment, the first group of cutting blades and the second group of cutting blades define a substantially hemispherical cutting area having an outer diameter.

In one other possible embodiment, rotation of the support member relative the housing moves at least the first group of cutting blades and changes the outer diameter of the substantially hemispherical cutting area.

In yet still another possible embodiment, the support member and the housing are concentrically aligned about a central axis, and the relative movement between the support member and the housing is about the central axis.

In yet another possible embodiment, the reamer further includes a connector that extends through the support member and engages the second group of cutting blades.

In still another possible embodiment, the housing includes a plurality of radial guides and each of the cutting blades in the first group of cutting blades extend between radial guides.

In another possible embodiment, the connector includes a cylindrical shaft that extends through an aperture in a platform supported by the support member and a threaded portion of the connector extends from the cylindrical shaft into a cylinder.

In still one other possible embodiment, the cylinder extends along the central axis and engages a distal end of the cylindrical shaft substantially at a confluence of the plurality of radial guides.

In yet still one other possible embodiment, a guide rail extends from at least one of the plurality of radial guides and into a slot at least partially defined by first and second flanges attached a corresponding cutting blade in the first group of cutting blades.

In yet another possible embodiment, the housing includes a substantially hemispherical portion and a cylindrical base.

In one other possible embodiment, each of the cutting blades in the first group of cutting blades are evenly spaced apart.

In still one other possible embodiment, the reamer further includes a spring-biased locking member, wherein engagement between the spring-biased locking member and the support member prevents relative movement between the support member and the housing.

In another possible embodiment, the housing includes a plurality of aperture to prevent clogging.

In accordance with another aspect of the invention, a reamer for forming cavities in bone is provided. The reamer may be broadly described as including a scroll gear extending from a support member for engaging a first group of cutting blades, a housing coaxially supported by the support member about a central axis, and a connector extending through the support member and scroll gear and engaging a second group of cutting blades. The first group of cutting blades extend at least partially through the housing and rotation of the support member and scroll gear causes the first group of cutting blades to move radially dependent upon a direction of rotation of the scroll gear.

In another possible embodiment, the first group of cutting blades and the second group of cutting blades define a substantially hemispherical cutting area having an outer diameter that changes with relative rotation between the support member and the housing.

In still another possible embodiment, a guide rail extends from at least one of a plurality of radial guides and into a slot at least partially defined by first and second flanges attached to a corresponding cutting blade in the first group of cutting blades.

In still yet one other embodiment, the housing includes a plurality of radial guides and each of the cutting blades in the first group of cutting blades extend between radial guides.

In another possible embodiment, a connector includes a cylindrical shaft that extends through an aperture in the support member and a threaded portion of the connector extends from the cylindrical shaft into a cylinder and engages the second group of cutting blades, and wherein the cylinder extends along the central axis and engages a distal end of the cylindrical shaft substantially at a confluence of the plurality of radial guides.

In the following description, there are shown and described several preferred embodiments of the reamer for forming cavities in bone. As it should be realized, the various reamers and methods are capable of other, different embodiments and their several details are capable of modification in various, obvious aspects all without departing from the syringes and methods as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the acetabular reamers and methods and together with the description serve to explain certain principles thereof. In the drawing figures:

FIG. 1 is a perspective view of a reamer for forming cavities in bone;

FIG. 2 is a side view of the reamer showing a position of one cutting blade adjacent a slot formed in a housing;

FIG. 3 is a perspective view of a standard power driver for attaching to the reamer.

FIG. 4 is a partial side view of the reamer with a connector extending through a support member and engaging a second group of cutting blades;

FIG. 5 is a top view of the reamer;

FIG. 6 is a perspective view of the reamer with the housing removed to show the support member, scroll gear extending from the support member, and the engagement between the first group of cutting blades and the scroll gear;

FIG. 7 is a bottom view of the scroll gear;

FIG. 8 is a perspective view of the of the housing of the reamer showing the relationship between the housing, guide rails, and the first group of cutting blades;

FIG. 9 is a partial side view of one of the cutting blades in the first group of cutting blades extending within a slot formed in the housing and engaging a guide rail; and

FIG. 10 is a bottom view of the reamer showing the spacing between blades within the first and second groups of blades.

Reference will now be made in detail to embodiments of the reamer, examples of which are illustrated in the accompanying drawing figures, wherein like numerals are used to represent like elements.

### DETAILED DESCRIPTION

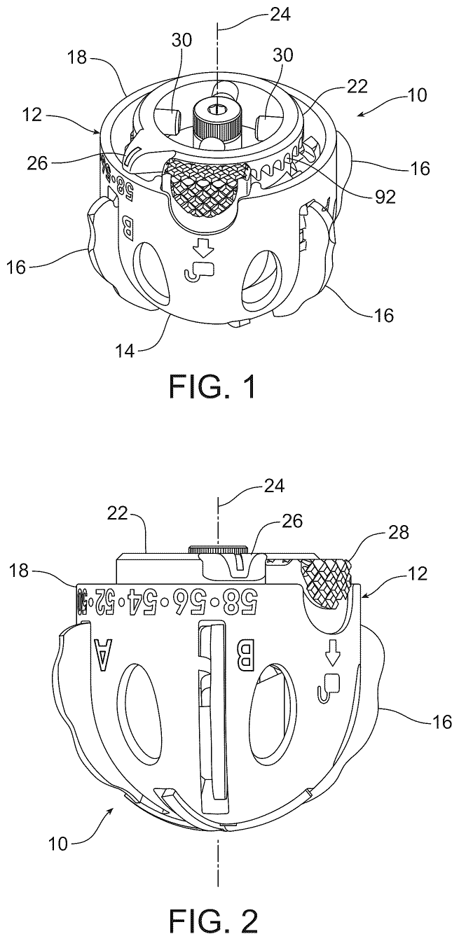

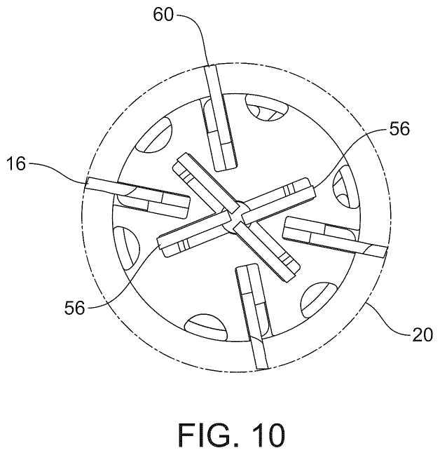

With reference to FIG. 1, an expandable acetabular reamer 10 for use in hip arthroplasty surgery is shown. The reamer 10 includes a housing 12 having a generally hemispherical head 14 and a cylindrical base 18. The housing 12 includes a plurality of generally oval-shaped apertures to allow debris to escape the housing during use to prevent clogging among other issues. As shown, a first group of cutting blades 16 at least partially extend through the housing 12 in a cutting position. In the described embodiment, the cutting blades 16 at least partially define a substantially hemispherical cutting area having an outer diameter 20 (shown in dashed line in FIG. 10) which hemispherical cutting area and outer diameter are adjustable in the described embodiment. The outer diameter, for example, is adjustable in 1 mm increments between a 48 mm diameter and a 58 mm diameter.

As shown in FIG. 2, numerals or other markings may be formed in or printed on the cylindrical base 18 to accommodate ease of selection of a desired cutting area outer diameter 20 during a procedure. Due to size limitations and a desire to maximize the size of the numerals to improve readability, the described embodiment only includes even numbers (e.g., “58,” “56,” etc.) that are separated by bullet points. The bullet points represent the odd numbers not shown between the displayed even numbers and allow the user to nonetheless select odd number diameters. Alternate embodiments may include more or fewer and even and/or odd numbers.

Returning to FIG. 1, a cylindrical support member 22 extends concentrically within the cutting head base 18 along a central axis 24. A pointer 26 extends from and is integrally formed with the support member 22 in the described embodiment and provides a visual indicator of a present position of the first group of cutting blades 16. Necessarily, the selected outer diameter of the substantially hemispherical cutting area (e.g., 52 mm) corresponds to a position of the cutting blades 16. As described in additional detail below, the support member 22 and pointer 26 rotate relative the cylindrical base 18 causing the cutting blades 16 to expand outwardly or retract inwardly depending on the desired outer diameter 20 of the hemispherical cutting area, and a locking member 28 secures the chosen relative positions.

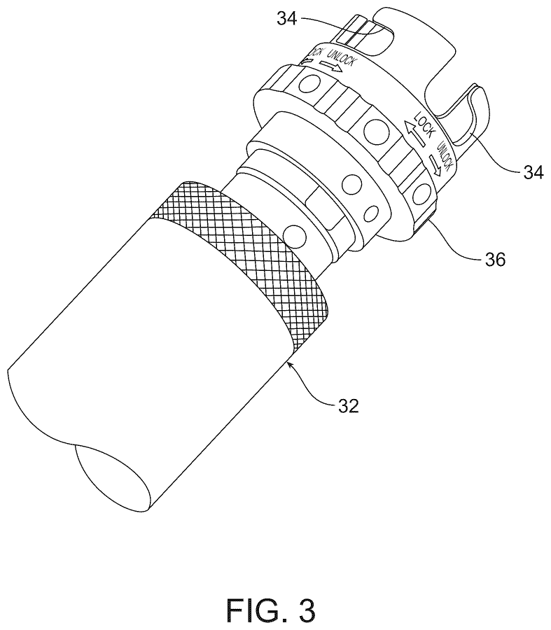

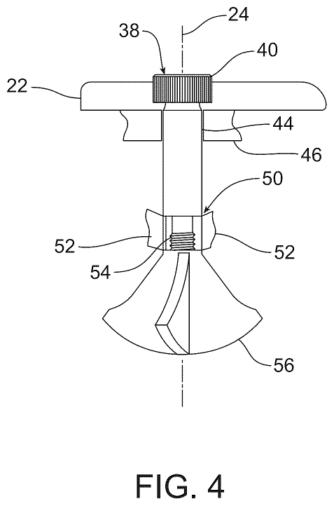

The support member 22 further includes a plurality of inwardly extending tabs or lugs 30, shown in FIG. 1, that engage corresponding slots 34 formed in a driver 32, partially shown in FIG. 3. A standard power driver, for example, may be utilized in surgical procedures to provide rotation to an attached tool such as reamer 10. Engagement between the slots 34 of the driver 32 and the lugs 30 of the reamer 10 ensures alignment and provides high torque transmission to the reamer with minimal complexity. The driver 32 includes a rotating head 36 wherein the engaging slots 34 are formed. In a typical operating manner, the lugs 30 of the support member 22 are positioned within the rotating head slots 34 and relative rotation between the support member and the rotating head 36 either locks the reamer 10 in position for use or unlocks the reamer for removal. Customary markings on the rotating head 36 of the driver 32 indicate “LOCK” and “UNLOCK” directions of rotation.

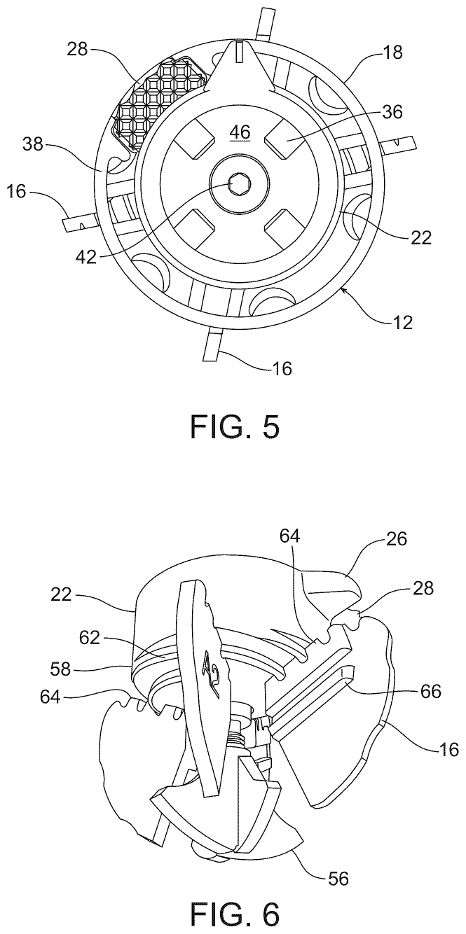

As best shown in FIGS. 4 and 5, a connector 38 extends concentrically within the cylindrical base 18 and the support member 22 along the central axis 24 and allows for assembly and disassembly of the reamer 10 to facilitate cleaning and sterilization, and blade replacement. A head 40 of the connector 38 is ridged to enhance grip, incorporates a drive recess 42 designed to receive an Allen wrench or similar tool in the described embodiment, and extends beyond a proximal end of the support member 22 as best shown in FIG. 4. In other embodiments, the head 40 may be knurled or the like and may or may not incorporate a drive recess or extend beyond the support member 22. Both features, however, provide ease of use for the surgeon or support staff by enhancing their ability to grasp and rotate the connector 38. In the described embodiment, the head 40 is sized to accommodate grasping even with gloved hands/digits.

The connector 38 further includes a cylindrical shaft 44 that extends through an aperture formed in a platform 46 supported by the cylindrical support member 22. Engagement between the cylindrical shaft 44 and platform 46 (best shown in FIG. 4) helps maintain the connector 38 and support member 22 in position about central axis 24. A distal end of the cylindrical shaft 44 engages or abuts a cylinder 50 extending along the central axis 24. As shown in FIG. 6, the cylinder 50 is positioned at a confluence of a plurality of radially extending guides 52 positioned within the housing 12. The connector 38 further includes a threaded shaft 54 extending distally beyond the cylindrical shaft 44. The threaded shaft 54 extends through an aperture defined by the cylinder 50 along the central axis 24 and, as best shown in FIG. 4, the threaded shaft 54 engages and secures a second group of cutting blades 56. The radial guide slots 52, their engagement with the first group of cutting blades 16, and the second group of cutting blades 56 are described in greater detail below.

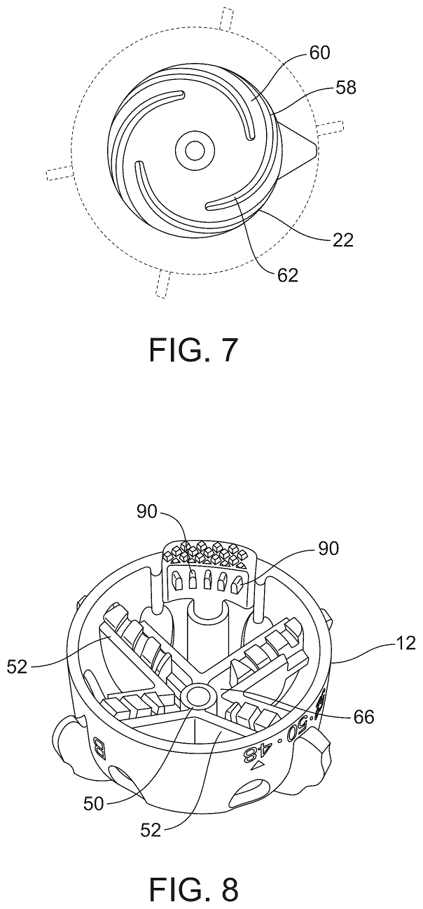

As best shown in FIG. 6, a scroll gear 58 extends distally from the cylindrical support member 22 into the cylindrical base 18 of the cutting head 14. The scroll gear 58 and cylindrical support member 22 are integrally formed in the described embodiment. As is known in the art and best shown in FIG. 7, the scroll gear 58 is generally a circular disk having a helical groove 60 formed into its face. More specifically, the helical groove 60 may be described as a continuous, Archimedean-like groove. Rather than having discrete gear teeth around its edge, the groove 60 forms a single wrapped tooth or edge surface 62 that runs from an inner portion of the of the circular disk out toward its perimeter.

When the surgeon rotates the support member 22 relative the cutting head 14, the scroll gear 58 is similarly rotated. Returning to FIG. 6, the edge surface 62 of the helical groove 60 engages one or more engagement teeth 64 extending from each of the individual blades of the first group of cutting blades 16. Rotation of the support member 22 and engagement between the scroll gear 58 and the engagement teeth 64 causes the cutting blades 16 to extend outwardly, thereby increasing the outer diameter 20 of the hemispherical cutting area, or retract inwardly, thereby decreasing the outer diameter, depending on the direction of rotation of the support member. Because the scroll gear groove 60 is continuous, each blade of the first group of blades 16 is driven simultaneously and the plurality of cutting blades move in unison, either inwardly or outwardly. In other words, the scroll gear 58 translates rotational motion into substantially synchronized linear motion for the cutting blades 16 in a controlled, uniform way.

As shown in FIG. 8, the cylinder 50 and the plurality of radially extending guides 52 define a plurality of slots 66 within which the cutting blades 16 reside, and restrain movement of the cutting blades. As a result, movement of each cutting blade 16 is generally limited to a radial direction relative the central axis 24. As noted above, engagement between the scroll gear 58 and the engagement teeth 64 causes the cutting blades 16 to extend radially outwardly or retract radially inwardly depending on the direction of rotation of the support member.

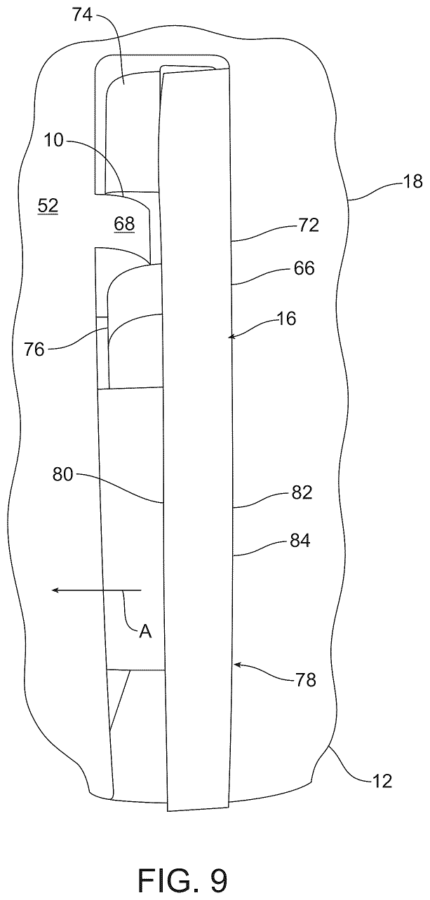

As shown in FIG. 9, movement of the cutting blade 16 is further restrained by a guide rail 68 extending from the radial guide 52 between an upper flange 74 and a lower flange 76. More specifically, the guide rail 68 extends into a guide track 70 defined by the upper flange 74 and the lower flange 76 which may engage the guide rail during operation to limit movement of the cutting blade 16. The upper and lower flanges 74, 76 are attached to or integrally formed with a body 72 of the cutting blade 16. The engagement teeth 64, noted above, extend from the upper flange 74 as shown in FIG. 6.

In the described embodiment, each cutting blade 16 includes the body 72 and a cutting edge 78. The chosen shape of the cutting edge 78 and angle relative the body 72 positions the cutting edge for use in preparing the cavity in the acetabulum for receiving the implant. As described in greater detail above, the cutting blades 16 at least partially define a substantially hemispherical cutting area having an outer diameter 20 which is adjustable in 1 mm increments. Rotation of the cylindrical support member 22 rotates the pointer 26 and causes rotation of the scroll gear 58 and radial movement of the cutting blades 16.

As shown throughout the figures and best in FIG. 9, the cutting blades in the first group of cutting blades 16 extend through associated slots 84 defined by the hemispherical head 14 and cylindrical base 18. A forward facing side 80 of the cutting edge 78 faces a direction of rotation (illustrated by action arrow A) of the reamer 10. A rearward facing side 82 of the cutting edge 78 is positioned adjacent a side wall 84 of the associated slot 88 formed in the hemispherical head 14 and cylindrical base 18. In this manner, the side wall 84 may provide additional support for the cutting blade 16 during use and engagement with the acetabular (not shown).

In addition, the cutting edge 78 is serrated to increase the sharpness of the edge in the described embodiment. In this embodiment, the cutting blades 16 include alternating cutting edges 78 such that a first serrated cutting edge 78 is followed by a second serrated cutting edge, and so on, so that there is overlap in the serrated areas to ensure a smooth overall result. Of course, in embodiments where the cutting blades do not have serrated cutting edges, alternating blades having different cutting edges would not be required.

As best shown in FIG. 10, there are four cutting blades 16 that form the first group of cutting blades. Although the cutting blades 16 may be equispaced and essentially ninety degrees apart from each another, the cutting blades in the described embodiment are offset so as not be at ninety degree angles from each other. This particular placement is intended to remove harmonic frequencies that can occur during use of the reamer 10. Of course, more or fewer cutting blades 16 may be utilized.

A second group of cutting blades 56 extend radially and generally distally through the hemispherical head 14 in a similar manner as the cutting blades 16. As best shown in FIG. 4, the second group of cutting blades 56 are integrally formed in the described embodiment. The threaded shaft 54 of the connector 38 engages the second group of cutting blades 56. The chosen shape of the second group of cutting blades 56 and extension beyond the hemispherical head 14 position the cutting blades for use in preparing the acetabulum cavity for receiving the implant. The second group of cutting blades 56 also form a portion of the substantially hemispherical cutting area described above. In the described embodiment, the second group of cutting blades 56 are fixed in position and a length of the individual blades is selected to avoid formation of a ridge between the first and second groups of cutting blades 16, 56 in operation. In addition, the second group of cutting blades 56 are also offset in the described embodiment to accommodate the offset position of the first group of cutting blades 16.

Returning to FIG. 1, the above-noted locking member 28 is provided to maintain the first and second groups of cutting blades 16, 56 in a selected position during use. In the described embodiment, the locking member 28 includes a thumb slide 86 that is biased toward an engaged or locking position. As best illustrated in FIG. 8, a stem extends from the thumb slide 86 into a cylinder 88 formed in the housing 12. A spring (not shown) positioned within the cylinder provides the noted bias in a known manner. One or more locking elements 90 extend from the thumb slide 86 and engage corresponding slots 92 formed in the cylindrical support member 22. The one or more locking elements are shown in FIG. 8 and the corresponding slots 92 are shown in FIG. 1.

In operation, the user engages the thumb slide 86 with sufficient force to overcome the bias and lower the slide to disengage the one or more locking elements 90 from corresponding slots 92. In this position, the user may further apply a rotational force on the cylindrical support member 22 to rotate the member to a next chosen position (e.g., 58 mm as shown in FIG. 2). When the support member 22 is rotated to the next chosen position, the thumb slide 86 is released allowing the spring bias to return the locking mechanism 28 to a locking position where the one or more locking elements 90 are re-engaged with the corresponding slots 92.

Last, it should be noted that the central connector 38 allows for assembly and disassembly of the reamer 10 primarily to facilitate cleaning and sterilization. Even more, however, disassembly of the reamer 10 allows for easy removal and replacement of the first and second cutting blade groups 16 and 56, as needed, in order to ensure optimal results.

The foregoing has been presented for purposes of illustration and description. It is not intended to be exhaustive or to limit the embodiments to the precise form of reamer disclosed. Obvious modifications and variations are possible in light of the above teachings. All such modifications and variations are within the scope of the appended claims when interpreted in accordance with the breadth to which they are fairly, legally and equitably entitled.

What is claimed:

1. A reamer for forming cavities in bone, comprising:
a housing;
a support member extending coaxially within and supporting the housing;
a scroll gear extending from the support member; and
a first group of cutting blades, each cutting blade in the first group of cutting blades including a plurality of teeth extending toward and engaging the scroll gear such that rotation of the scroll gear causes each cutting blade in the first group of cutting blades to move dependent upon a direction of rotation of the scroll gear,
wherein each cutting blade in the first group of cutting blades at least partially extends through the housing in a cutting position.

2. The reamer for forming cavities in bone of claim 1, further comprising a second group of cutting blades wherein each of the cutting blades in the second group of cutting blades extends through the housing.

3. The reamer for forming cavities in bone of claim 2, wherein each of the cutting blades in the second group of cutting blades is fixed in position relative the housing.

4. The reamer for forming cavities in bone of claim 2, wherein the first group of cutting blades and the second group of cutting blades define a substantially hemispherical cutting area having an outer diameter.

5. The reamer for forming cavities in bone of claim 4, wherein rotation of the support member relative the housing moves at least the first group of cutting blades and changes the outer diameter of the substantially hemispherical cutting area.

6. The reamer for forming cavities in bone of claim 5, wherein the support member and the housing are concentrically aligned about a central axis, and the relative movement between the support member and the housing is about the central axis.

7. The reamer for forming cavities in bone of claim 6, wherein a connector extends through the support member and engages the second group of cutting blades.

8. The reamer for forming cavities in bone of claim 7, wherein the housing includes a plurality of radial guides and each of the cutting blades in the first group of cutting blades extend between radial guides.

9. The reamer for forming cavities in bone of claim 8, wherein the connector includes a cylindrical shaft that extends through an aperture in a platform supported by the support member and a threaded portion of the connector extends from the cylindrical shaft into a cylinder.

10. The reamer for forming cavities in bone of claim 9, where the cylinder extends along the central axis and engages a distal end of the cylindrical shaft substantially at a confluence of the plurality of radial guides.

11. The reamer for forming cavities in bone of claim 8, wherein a guide rail extends from at least one of the plurality of radial guides and into a slot at least partially defined by first and second flanges attached to a corresponding cutting blade in the first group of cutting blades.

12. The reamer for forming cavities in bone of claim 1, wherein the housing includes a substantially hemispherical portion and a cylindrical base.

13. The reamer for forming cavities in bone of claim 1, wherein each of the cutting blades in the first group of cutting blades are evenly spaced apart.

14. The reamer for forming cavities in bone of claim 1, further comprising a spring-biased locking member, wherein engagement between the spring-biased locking member and the support member prevents relative movement between the support member and the housing.

15. The reamer for forming cavities in bone of claim 1, wherein the housing includes a plurality of aperture to prevent clogging.

16. A reamer for forming cavities in bone, comprising:
a scroll gear extending from a support member for engaging a first group of cutting blades;
a housing coaxially supporting the support member about a central axis; and
a connector extending through the support member and scroll gear and engaging a second group of cutting blades,
wherein the first group of cutting blades extend at least partially through the housing and rotation of the support member and scroll gear causes the first group of cutting blades to move radially dependent upon a direction of rotation of the scroll gear.

17. The reamer for forming cavities in bone of claim 16, wherein the first group of cutting blades and the second group of cutting blades define a substantially hemispherical cutting area having an outer diameter that changes with relative rotation between the support member and the housing.

18. The reamer for forming cavities in bone of claim 16, wherein a guide rail extends from at least one of a plurality of radial guides and within a slot at least partially defined by first and second flanges attached to a corresponding cutting blade in the first group of cutting blades.

19. The reamer for forming cavities in bone of claim 16, wherein the housing includes a plurality of radial guides and each of the cutting blades in the first group of cutting blades extend between radial guides.

20. The reamer for forming cavities in bone of claim 16, wherein a connector includes a cylindrical shaft that extends through an aperture in the support member and a threaded portion of the connector extends from the cylindrical shaft into a cylinder and engages the second group of cutting blades, and wherein the cylinder extends distally along the central axis and engages a distal end of the cylindrical shaft substantially at a confluence of the plurality of radial guides.
